(19) United States
(12) Patent Application Publication
(10) Pub. No.: US 2026/0294508 A1
(43) Pub. Date: Oct. 1, 2026

(54) RF POWER TIME MULTIPLEX STRATEGY TO DELIVER TWO INDEPENDENT BVN THERAPIES SIMULTANEOUSLY FROM SINGLE POWER SOURCE

(71) Applicant: Boston Scientific Scimed, Inc., Maple Grove, MN (US)

(72) Inventors: Jin Yu, Valley Village, CA (US); Alexander Pruitt, San Jose, CA (US)

(21) Appl. No.: 19/531,313

(22) Filed: Feb. 5, 2026

Related U.S. Application Data
(60) Provisional application No. 63/754,315, filed on Feb. 5, 2025.

(51) Int. Cl. A61B 18/12; A61B 18/00

(52) U.S. Cl. CPC A61B 18/1206; A61B 2018/00577; A61B 2018/00702; A61B 2018/00726; A61B 2018/00761; A61B 2018/00767; A61B 2018/00779; A61B 2018/00875

(57) ABSTRACT

Tissue ablation system that delivers independent therapies to multiple channels using a single radiofrequency power source through power time multiplexing. The system includes a radiofrequency generator having a controller that alternates power delivery between channels within defined time cycles. The controller determines power demands for each channel, calculates duty cycles for power delivery, and sets a common voltage amplitude based on the highest power requirements between channels. The system can operate in fixed time allocation mode where cycle time is evenly split between channels, or in dynamic allocation mode where duty cycles are adjusted to optimize voltage amplitude while maintaining required power delivery to each channel. The system enables independent therapy delivery through multiple channels while efficiently using a single power source.

### CROSS REFERENCE TO RELATED APPLICATIONS

This application claims priority to U.S. Provisional Patent Application No. 63/754,315 entitled “RF POWER TIME MULTIPLEX STRATEGY TO DELIVER TWO INDEPENDENT BVN THERAPIES SIMULTANEOUSLY FROM SINGLE POWER SOURCE,” filed Feb. 5, 2025, which is hereby incorporated by reference in its entirety.

### TECHNICAL FIELD

The present invention relates to tissue ablation systems and methods of operating tissue ablation systems. More specifically, the present invention relates to radiofrequency time multiplexing strategies for delivering multiple independent therapies from a single radiofrequency generator during surgical procedures.

### BACKGROUND

Radiofrequency energy has been used to ablate tissue at various locations within the body. For example, radiofrequency probes have been used to ablate tissue within vertebral bodies to treat chronic back pain, to ablate nerves to treat facet joint pain, or to ablate tissue within joints to treat joint pain within the knees, shoulders, elbows, hips, or ankles.

A multichannel radiofrequency tissue ablation system includes multiple channels and can perform simultaneous thermal ablations using more than one monopolar or bipolar radiofrequency probe. Traditional multichannel radiofrequency tissue ablation systems with a single radiofrequency generation source have limitations in delivering independent therapies, as they typically can only provide the same therapy with the same parameter settings across all channels. This means that different probes cannot be independently controlled to deliver different therapy parameters when operating simultaneously.

Therefore, there is a need to develop methods and systems that enable independent therapy delivery to multiple channels from a single radiofrequency source while maintaining precise control over the therapy parameters for each channel.

### SUMMARY

The present invention addresses the need to deliver independent radiofrequency therapies to multiple channels by utilizing time multiplexing methods and techniques based on selected procedure operational parameters for controlling radiofrequency energy delivery to radiofrequency probes that are coupled to a radiofrequency generator. The time multiplexing techniques described herein enable the tissue ablation system to implement controlled independent therapies through a single power source (e.g., RF generator), providing flexible and independent control of therapy parameters for each channel. In one aspect, the tissue ablation system of the present invention provides selected time multiplexing techniques including one or more of: dynamic duty cycle adjustment, common voltage amplitude determination, and optimization of power delivery timing between channels. The system can dynamically assess and adjust power supply timing based on real-time demands of each channel.

Another aspect of the present invention may include a tissue ablation system comprising a radiofrequency generator having a power source and a controller operatively coupled to the power source and having a processor and a memory element, a first radiofrequency probe having at least one electrode and electrically coupled to the radiofrequency generator to form a first channel, and a second radiofrequency probe having at least one electrode and electrically coupled to the radiofrequency generator to form a second channel. The power source can be configured to deliver radiofrequency power to the one or more electrodes of each radiofrequency probe sufficient to provide controlled heating of tissue surrounding the electrodes during a surgical procedure. The processor can execute program instructions to determine power demands for each channel and implement controlled power delivery between the two channels using a common voltage amplitude. The system implements various time multiplexing techniques that can include determining minimum voltage amplitudes that satisfy both channels' requirements, dynamically adjusting duty cycles based on channel power demands, and optimizing timing to enable effective therapy delivery while maintaining independent control of each channel.

In Example 1, a tissue ablation system comprising a radiofrequency generator having a power source and a controller that is operatively coupled to the power source and having a processor and a memory element, a first radiofrequency probe having at least one electrode and electrically coupled to the radiofrequency generator to form a first channel, and a second radiofrequency probe having at least one electrode and electrically coupled to the radiofrequency generator to form a second channel. The power source can be configured to deliver radiofrequency power to the electrodes of the first and second radiofrequency probes. The processor is also configured to execute program instructions to determine a first power demand for the first channel and a second power demand for the second channel and to implement a power-time multiplexing technique. The power time multiplexing technique allocates a first duty cycle to the first channel and a second duty cycle to the second channel, and determines a common radiofrequency voltage for each of the first channel and the second channel during the first and second duty cycles, respectively, based on whichever of the first power demand or the second power demand has a higher radiofrequency voltage, and deliver radiofrequency power at the common radiofrequency voltage between the first channel and the second channel within each of the first and second duty cycles, respectively, when each of the first and second channels is in an activation state.

In Example 2, the tissue ablation system of Example 1 wherein the first duty cycle and the second duty cycle have the same time duration.

In Example 3, the tissue ablation system of any of Examples 1-2, wherein during the power-time multiplexing technique the processor is further configured to: determine a first average power for the first channel according to the formula P1=(V1×V1/R1)×D1, wherein V1 is voltage, R1 is impedance, and D1 is duty cycle for the first channel; and determine a second average power for the second channel according to the formula P2=(V2×V2/R2)×D2, where V2 is voltage, R2 is impedance, and D2 is duty cycle for the second channel.

In Example 4, the tissue ablation system of any of Examples 1-3, wherein the processor is further configured to implement a power-time rebalancing technique by adjusting a duration of each of the first duty cycle and the second duty cycle based on the respective power demands of the first channel and the second channel.

In Example 5, the tissue ablation system of Example 4, wherein the processor is configured to, during the power-time rebalancing technique: determine a new first duty cycle (Dnew1) for the first channel according to Dnew1=Varg1×Varg1/(Varg1×Varg1+Varg2×Varg2), wherein Varg1 and Varg2 are adjusted voltages for the first and second channels, respectively; and determine a new radiofrequency voltage amplitude (Vnew) during the new first duty cycle according to Vnew=Varg1/sqrt(Dnew1).

In Example 6, the tissue ablation system of any of Examples 4-5, wherein the processor is further configured to allocate up to 92% of a cycle period between the first and second channels.

In Example 7, the tissue ablation system of any of Examples 1-6, wherein the processor is further configured to: monitor an impedance of each of the first and second channels during the respective activation state; and adjust an amplitude of the common radiofrequency voltage based on the impedance.

In Example 8, the tissue ablation system of any of Examples 1-7, wherein the processor is configured to adjust a duration of one of the first duty cycle and the second duty cycle during the activation state to maintain equivalent radiofrequency power delivery thereto while reducing an amplitude of the common radiofrequency voltage.

In Example 9, the tissue ablation system of any of Examples 1-8, wherein the first duty cycle and the second duty cycle define a cycle time, and wherein, if one of the first and second channels requires minimal or no power, the processor is configured to: allocate a majority of the cycle time to the other one of the first and second channels; and reduce an amplitude of the common radiofrequency voltage.

In Example 10, the tissue ablation system of any of Examples 1-9, wherein the processor is further configured to determine a minimum common radiofrequency voltage capable of satisfying the first power demand and the second power demand of the first and second channels, respectively.

In Example 11, the tissue ablation system of any of Examples 1-10, wherein the controller is further configured to: maintain the determinations of the first and second power demands of the first and second channels, respectively; and continuously adjust the first and second duty cycles based on changes in the first and second power demands.

In Example 12, the tissue ablation system of any of Examples 1-11, wherein the controller is configured to determine the first and second duty cycles by: determining which of the first and second channels requires a higher power demand; allocating a larger portion of the cycle time to the duty cycle of the channel requiring the higher power demand; and allocating a remaining portion of the cycle time to the other one of the first and second channels.

In Example 13, the tissue ablation system of any of Examples 1-12, wherein the controller is configured to determine a minimum common radiofrequency voltage amplitude by: determining the radiofrequency voltage requirements for each of the first and second channels based on associated power demands and allocated with the first and second duty cycles; selecting a higher one of the radiofrequency voltage requirements as the common radiofrequency voltage amplitude; and adjusting the first and second duty cycles to maintain power delivery at the selected voltage amplitude.

In Example 14, the tissue ablation system of any of Examples 1-13, wherein the controller is configured to monitor power delivery to each channel during the respective activation state; detect when one of the first and second channels requires minimal power delivery; and reallocate the first and second duty cycles to minimize an amplitude of the common radiofrequency voltage.

In Example 15, the tissue ablation system of any of Examples 1-14, wherein the radiofrequency power is sufficient to provide controlled heating of tissue surrounding the electrodes during a surgical procedure.

In Example 16, a tissue ablation system comprising a radiofrequency generator having a power source and a controller operatively coupled to the power source and having a processor and a memory element. The tissue ablation system further comprising a first radiofrequency probe having at least one electrode and electrically coupled to the radiofrequency generator to form a first channel and a second radiofrequency probe having at least one electrode and electrically coupled to the radiofrequency generator to form a second channel. The power source is configured to deliver radiofrequency power to the electrodes of the first and second radiofrequency probes sufficient to provide controlled heating of tissue surrounding the electrodes during a surgical procedure. The processor is further configured to execute program instructions to determine a first power demand for the first channel and a second power demand for the second channel, implement a power-time multiplexing technique by:
allocating a first duty cycle to the first channel and a second duty cycle to the second channel; and determining a common radiofrequency voltage for each of the first channel and the second channel during the first and second duty cycles, respectively, based on whichever of the first power demand or the second power demand has a higher radiofrequency voltage; and deliver radiofrequency power at the common radiofrequency voltage between the first channel and the second channel within each of the first and second duty cycles, respectively, when each of the first and second channels is in an activation state.

In Example 17, the tissue ablation system of Example 16, wherein the first duty cycle and the second duty cycle have the same time duration.

In Example 18, the tissue ablation system of Example 17, wherein during the power-time multiplexing technique the processor is further configured to: determine a first average power for the first channel according to the formula P1=(V1×V1/R1)×D1, wherein V1 is voltage, R1 is impedance, and D1 is duty cycle for the first channel; and determine a second average power for the second channel according to the formula P2=(V2×V2/R2)×D2, where V2 is voltage, R2 is impedance, and D2 is duty cycle for the second channel.

In Example 19, the tissue ablation system of Example 16, wherein the processor is further configured to implement a power-time rebalancing technique by adjusting a duration of each of the first duty cycle and the second duty cycle based on the respective power demands of the first channel and the second channel.

In Example 20, the tissue ablation system of Example 19, wherein the processor is configured to, during the power-time rebalancing technique: determine a new first duty cycle (Dnew1) for the first channel according to Dnew1=Varg1×Varg1/(Varg1×Varg1+Varg2×Varg2), wherein Varg1 and Varg2 are adjusted voltages for the first and second channels, respectively; and determine a new radiofrequency voltage amplitude (Vnew) during the new first duty cycle according to Vnew=Varg1/sqrt(Dnew1).

In Example 21, the tissue ablation system of Example 20, wherein the processor is further configured to allocate up to 92% of a cycle period between the first and second channels.

In Example 22, the tissue ablation system of Example 16, wherein the processor is further configured to: monitor an impedance of each of the first and second channels during the respective activation state; and adjust an amplitude of the common radiofrequency voltage based on the impedance.

In Example 23, the tissue ablation system of Example 16, wherein the processor is configured to adjust a duration of one of the first duty cycle and the second duty cycle during the activation state to maintain equivalent radiofrequency power delivery thereto while reducing an amplitude of the common radiofrequency voltage.

In Example 24, the tissue ablation system of Example 16, wherein the first duty cycle and the second duty cycle define a cycle time, and wherein, if one of the first and second channels requires minimal or no power, the processor is configured to: allocate a majority of the cycle time to the other one of the first and second channels; and reduce an amplitude of the common radiofrequency voltage.

In Example 25, the tissue ablation system of Example 16, wherein the processor is further configured to determine a minimum common radiofrequency voltage capable of satisfying the first power demand and the second power demand of the first and second channels, respectively.

In Example 26, the tissue ablation system of Example 16, wherein the controller is further configured to: maintain the determinations of the first and second power demands of the first and second channels, respectively; and continuously adjust the first and second duty cycles based on changes in the first and second power demands.

In Example 27, the tissue ablation system of Example 16, wherein the controller is configured to determine the first and second duty cycles by: determining which of the first and second channels requires a higher power demand; allocating a larger portion of the cycle time to the duty cycle of the channel requiring the higher power demand; and allocating a remaining portion of the cycle time to the other one of the first and second channels.

In Example 28, the tissue ablation system of Example 16, wherein the controller is configured to determine a minimum common radiofrequency voltage amplitude by: determining the radiofrequency voltage requirements for each of the first and second channels based on associated power demands and allocated with the first and second duty cycles; selecting a higher one of the radiofrequency voltage requirements as the common radiofrequency voltage amplitude; and adjusting the first and second duty cycles to maintain power delivery at the selected voltage amplitude.

In Example 29, the tissue ablation system of Example 16, wherein the controller is configured to monitor power delivery to each channel during the respective activation state; detect when one of the first and second channels requires minimal power delivery; and reallocate the first and second duty cycles to minimize an amplitude of the common radiofrequency voltage.

In Example 30, the tissue ablation system of Example 16, wherein the controller is configured to determine average powers for the first and second channels based on the common radiofrequency voltage and their respective duty cycles.

In Example 31, the tissue ablation system of Example 16, wherein the controller is configured to maintain equivalent power delivery while reducing the common radiofrequency voltage by increasing the duty cycle allocated to the channel requiring higher power.

In Example 32, a computer-implemented method of allocating power in a tissue ablation system is provided having a radiofrequency generator and first and second radiofrequency probes forming respective first and second channels. The method comprising executing, by a processor of a controller operatively coupled to the radiofrequency generator, program instructions stored in a memory element to determine a first power demand for the first channel and a second power demand for the second channel; implement a power-time multiplexing technique by: allocating a first duty cycle to the first channel and a second duty cycle to the second channel; and determining a common radiofrequency voltage for each of the first channel and the second channel during the first and second duty cycles, respectively, based on whichever of the first power demand or the second power demand has a higher radiofrequency voltage; and deliver radiofrequency power at the common radiofrequency voltage between the first channel and the second channel within each of the first and second duty cycles, respectively, when each of the first and second channels is in an activation state.

In Example 33, the computer-implemented method of Example 32, wherein during the power-time multiplexing technique the processor is further configured to: determine a first average power for the first channel according to the formula P1=(V1×V1/R1)×D1, wherein V1 is voltage, R1 is impedance, and D1 is duty cycle for the first channel; and determine a second average power for the second channel according to the formula P2=(V2×V2/R2)×D2, where V2 is voltage, R2 is impedance, and D2 is duty cycle for the second channel.

In Example 34, the computer-implemented method of Example 32, wherein the processor is further configured to implement a power-time rebalancing technique by adjusting a duration of each of the first duty cycle and the second duty cycle based on the respective power demands of the first channel and the second channel.

In Example 35,, the computer-implemented method of Example 34, wherein the processor is configured to, during the power-time rebalancing technique: determine a new first duty cycle (Dnew1) for the first channel according to Dnew1=Varg1×Varg1/(Varg1×Varg1+Varg2×Varg2), wherein Varg1 and Varg2 are adjusted average voltages for the first and second channels, respectively; and determine a new radiofrequency voltage amplitude (Vnew) during the new first duty cycle according to Vnew=Varg1/sqrt(Dnew1).

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic block diagram of an exemplary multichannel radiofrequency tissue ablation system in accordance with the teachings of the present invention;

FIG. 2 illustrates conventional power profiles of two independent therapies with their own dedicated power source;

FIG. 3 illustrates a power time multiplex technique showing alternating delivery of power between two channels with a common voltage amplitude utilizing a single RF generator in accordance with the teachings of the present invention;

FIGS. 4A-4C are schematic diagrams illustrating power rebalancing between two channels during time multiplexing according to the teachings of the present invention; and

FIGS. 5A-5C are schematic diagrams illustrating illustrative voltage delivery patterns for different operating conditions over two channels according to the teachings of the present invention.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

A radiofrequency ablation (RFA) procedure is a minimally invasive medical procedure that is used to ablate targeted tissue at a surgical site, and typically involves using one or more energy delivery devices (e.g., radiofrequency (RF) probes) having one or more electrodes associated therewith. The radiofrequency probe can be coupled to one or more radiofrequency (RF) generators to deliver power in the form of radiofrequency energy to the targeted tissue via the electrodes. The radiofrequency energy generates localized heat which is used to target and destroy (e.g., ablate) the targeted tissue. In pain management, the targeted tissue can be nerve tissue responsible for sending pain signals.

The RFA procedure can be utilized to ablate different types of tissue, such as nerve tissue, adipose tissue, muscle tissue, and the like. The RFA procedure can be performed at various locations on an external surface of a subject or patient or within the subject. The RFA procedure performed within the subject can be performed in a minimally invasive manner (e.g., via a percutaneous, laparoscopic, endoscopic, or intravascular approach) or in a more invasive manner (e.g., via an open surgical procedure). For example, Applicant's existing technology (the Intracept® procedure by Relievant® Medsystems, Inc.) offers a safe and effective minimally invasive procedure that targets the basivertebral nerve (BVN) for the relief of chronic low back pain that originates at least partly from one or more endplates of one or more vertebral bodies. The Intracept® procedure involves application of radiofrequency energy from a radiofrequency generator using a bipolar radiofrequency probe, where the applied radiofrequency energy is sufficient to ablate the basivertebral nerve within the vertebral body. The procedure can be performed in multiple different vertebral bodies sequentially or simultaneously using a single radiofrequency probe or using multiple radiofrequency probes coupled to a single generator. The basivertebral nerve trunk can then be ablated by the RF probe. Other intraosseous nerves within the vertebral body that innervate the endplates and/or intervertebral disc can also be targeted and ablated.

The RFA procedure is typically performed using a surgical introducing or access assembly that can include a hollow cannula that forms a sheath and a stylet that is disposed within the cannula until a distal tip of the stylet extends beyond the cannula. The stylet adds rigidity and support to the cannula when inserted into the patient. The stylet helps the surgeon guide the assembly to the surgical site. Once the cannula is in position at the surgical site, then the stylet is removed from the cannula and the RF probe is inserted therein. The RF probe can then deliver RF energy to the targeted tissue at the surgical site. In a bipolar RF probe arrangement, the probe can include a pair of electrodes, including a first active energy delivering electrode and a second return electrode. According to another arrangement, the RF probes can be configured in a monopolar arrangement, where an RF probe employs an active energy delivering electrode and a separate device or element can be used as a return. The return can be configured as a return pad coupled to the subject or a return electrode disposed on a separate RF probe. For example, a first RF probe can be used that includes the active energy delivering electrode and a second RF probe can be used that includes a return electrode. The RF probe can also include additional structure, including for example temperature sensors (e.g., thermocouples or thermistors), that can be employed to sense or detect the temperature at the surgical site during the surgical procedure. The sensed or detected temperature can provide important information to the surgeon about the ablation procedure. The RFA procedure contemplates positioning the two electrodes of the one or more radiofrequency probes within the vertebral body. The RF generator supplies power to the active electrode of the RF probe at a selected power or voltage level and for a time sufficient to create a desired lesion within the vertebral body sufficient to ablate the basivertebral nerve within the vertebral body. In some implementations, the step of applying power to the RF probe includes supplying power for a selected duration or period of time (e.g., 1 minute to 1 hour or for any time within this range) to achieve a selected temperature at the surgical site. The temperature of the thermal energy supplied by the RF probe to the targeted tissue can range from between about 50° C. and about 115° C. (e.g., from about 70° C. to about 90° C., from about 75° C. to about 90° C., from about 83° C. to about 87° C., from about 80° C. to about 100° C., from about 85° C. to about 95° C., from about 90° C. to about 110° C., from about 95° C. to about 115° C., or overlapping ranges thereof). The temperature ramp-up during the surgical procedure can range from between about 0.1-5 degrees Celsius/second (e.g., 0.1-1.0 degrees Celsius/second, 0.25 to 2.5 degrees Celsius/second, 0.5-2.0 degrees Celsius/second, 1.0-3.0 degrees Celsius/second, 1.5-4.0 degree Celsius/second, 2.0-5.0 degrees Celsius/second). The time of treatment can range from between about 10 seconds and about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy can be delivered as an alternative to or in sequence with continuous energy. For radiofrequency energy, the energy applied by the RF generator can range from between about 350 kHz and about 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz±5 kHz, 475 kHz±5 kHz, 487 kHz±5 kHz). The power of the radiofrequency energy generated by the RF generator can range from between about 5 W and about 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges).

Although described primarily in connection with procedures for the treatment within the spine, the systems and methods of the present invention can also be used for radiofrequency tissue ablation procedures intended to treat ailments or conditions other than those associated with the spine or back pain. The procedures can involve ablation of nerves outside of bones but related to the spine (e.g., sacroiliac joints, facet joints, etc.). Target treatment locations within bones other than vertebral bodies may also be accessed. For example, target treatment locations within a humerus, radius, femur, tibia, calcaneus, tarsal bones, hips, knees, phalanges, and/or other orthopedic targets can be accessed. The ablation procedures can include, for example, ablation of nerves within or surrounding other bones other than the vertebral column, cardiac tissue ablation for treatment of atrial fibrillation or other abnormal heart rhythms irregularities, tumor ablation at any location within the body (e.g., within bones, lungs, breasts, thyroids, livers, or other organs or tissues), peripheral nerve ablation, pulmonary artery ablation, renal denervation procedures, uterine fibroid ablation, endometrial ablation, and/or the like. The systems and methods described herein may be used in connection with any radiofrequency procedure during which impedance (e.g., tissue impedance or impedance between two electrodes) is monitored.

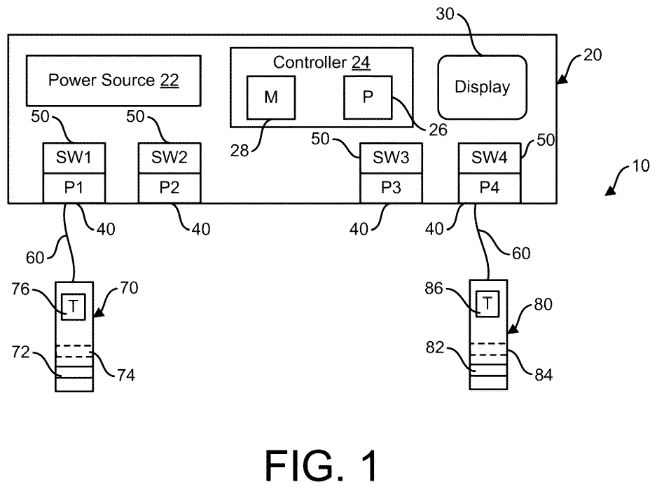

FIG. 1 is a schematic block diagram of a tissue ablation system 10 suitable for use with the present invention. The tissue ablation system includes a generator, such as a radiofrequency generator 20, that is coupled to one or more radiofrequency probes 70, 80. The RF generator 20 can be electrically and communicatively coupled to the RF probes 70, 80 via any suitable conduit 60. The conduits 60 can facilitate bidirectional communication of electrical signals and data or instructions between the RF generator 20 and the RF probes 70, 80. For example, the conduits 60 can include one or more electrical wires or lines (not shown) suitable for conveying the electrical signals. The generator can also be any other type of energy source suitable for supplying ablative energy to the probes. According to one embodiment, the illustrated RF generator 20 can include a power source 22 that can be configured to generate and supply radiofrequency power or energy to the RF probes 70, 80 via the conduits 60 at a desired frequency and power level. The RF generator 20 can also include a controller 24 that includes for example a processor (e.g., CPU) 26 suitable for processing and executing applications and instructions that are stored in a memory element 28. The processor 26 can be any suitable type of processor, such as a special purpose processor, and the memory element 28 can be any suitable type of memory. The processor 26 can be implemented in hardware, software, firmware, or any suitable combination thereof, and can be configured to execute program instructions stored in the memory element 28. The processor 26 and the memory element 28 are shown herein as forming part of the controller 24, but one of ordinary skill in the art will readily recognize that the processor 26 and the memory 28 can form part of other components of the RF generator 20. For the sake of simplicity, only one processor 26 and memory element 28 are shown, although multiple processors and memory elements can also be employed in the RF generator 20. The controller 24 can be configured as described herein, and at least is configured to monitor temperature, power, and/or impedance in connection with a tissue ablation procedure performed by the tissue ablation system 10. The RF generator 20 can further include a user interface or display element 30 suitable for displaying information or instructions to a user (e.g., surgeon) via a series of user interfaces or for allowing the user to provide instructions to the RF generator. As such, the display element 30 can employ a user interface generator 32 for generating the user interfaces based on programmatic instructions stored in the memory element 28 and processed by the processor 28 or by a processor forming part of the display element 30. The display element 30 can be configured to display information to the user. For example, during startup and use, the current status of the RF generator 20 and energy delivery or treatment parameters can be displayed on the display element 30 via one or more user interfaces. During energy delivery, the display element 30 can be configured to display desired treatment time, remaining treatment time, temperature, impedance, and power information (alphanumerically and/or graphically). For example, graphical representations of power vs. time and impedance vs. time can be displayed. The display element 30 can also be separate from the RF generator 20 and need not form part of the generator. Although not shown, the tissue ablation system 10 can also employ additional accessory devices, such as suitable user input devices including a keyboard, mouse, trackpad, voice-activated input device, and the like.

The illustrated RF generator 20 can also have formed therein a plurality or series of connector ports 40 that allow the RF probes 70, 80 to be coupled to the generator. The RF generator 20 can have any selected number of connector ports 40. In the illustrated embodiment, the RF generator 20 includes four probe connector ports 40A and a ground connector port 40B. The connector ports 40 can have any selected size, shape, and configuration. According to one embodiment, the connector ports 40 can employ a series of electrical pins that connect to RF probes and a ground pad and allow electrical energy and data to be exchanged therebetween. For example, a first electrical pin (or multiple electrical pins) can be coupled to the RF probe so as to deliver RF power to an energy delivering electrode when placed thereon and a second electrical pin can communicate with a return electrode when placed thereon. A third electrical pin can be employed to communicate with a temperature sensor in the RF probe and a fourth electrical pin can be used to exchange data between the RF generator and the RF probe. Those of ordinary skill in the art will readily recognize that any suitable type (e.g., Lemo or Din type connectors), arrangement, and number of electrical connectors can be employed in the connector ports 40.

The illustrated RF generator 20 can also employ a switching element module or a series of discrete switching elements 50 that can be used to help control the generation, modulation, and delivery of RF energy. The switching elements 50 help ensure the precise delivery of energy to the target tissue via the RF probes while maintaining system safety and functionality. The switching elements 50 can also control the RF energy delivered to the active energy delivering electrode of the RF probe through an associated connector port 40. For the sake of simplicity, a switching element 50 is shown coupled to and disposed in electrical communication with each illustrated connector port 40. Those of ordinary skill in the art will readily recognize that a separate switching element module that is arranged in electrical communication with each of the connector ports 40 can also be employed. The illustrated switching elements 50 can also be positioned and arranged to manage the activation of circuits connected to sensors in the RF probes 70, 80, such as temperature sensors or impedance measurement systems, and to manage the activation of the ground connector port 40B. The switching elements 50 can also be arranged to route sensor signals to specific monitoring circuits in the controller 24 for processing. The switching elements 50 can also serve to verify proper grounding or return electrode connection before enabling RF output to ensure patient safety. The switching elements 50 can be any suitable type of switching element, such as MOSFETs, IGBTs, Pin diodes, mechanical and solid-state relays, and the like.

The illustrated tissue ablation system 10 can also employ one or more RF probes. For the sake of simplicity and for purposes of explanation, a pair of RF probes 70, 80 are shown connected to the RF generator 20. The RF probes 70, 80 can be the same or can be different depending upon the selected type of surgical procedure being performed and/or the purpose and function of the RF probe. The RF probe 70 has a main body having an outer surface having one or more electrodes mounted thereon. In a bipolar RF probe arrangement, the RF probe 70 can have a first active energy delivering electrode 72 and a return electrode 74 disposed on an outer surface of the main body. The probe can also include a temperature sensor 76 for sensing or measuring temperature at the surgical site. The electrodes and the temperature sensor 76 can be coupled to the RF generator 20 via electrical leads that pass through the conduit 60. If the RF probe 70 is constructed as a monopolar device, then the probe can include a single active electrode 72, which can function as the active energy delivering electrode or as the return electrode. In this type of arrangement, the second electrode 74 (in dashed lines) is either inactivated or not present. Similarly, the RF probe 80 has a main body having an outer surface having one or more electrodes mounted thereon. In a bipolar RF probe arrangement, the RF probe 80 can also have a first active energy delivering electrode 82 and a return electrode 84. The probe can also include a temperature sensor 86. The electrodes and the sensor can also be coupled to the RF generator 20 via electrical leads that pass through the conduit 60. If the RF probe 80 is constructed as a monopolar device, then the probe can also include a single active electrode 82, which can function as the active energy delivering electrode or as the return electrode. In this type of arrangement, the second electrode 84 is either inactivated or not present. Each RF probe that is coupled to a connector port 40 of the RF generator 20 forms in essence a channel. As such, multiple RF probes coupled to multiple connector ports 40 of the RF generator 20 can form multiple channels.

The systems and methods described herein are generally aimed at providing time multiplexing approaches to enable independent therapy delivery in multichannel radiofrequency tissue ablation systems with a single radiofrequency generation source. Time multiplexing refers to a power delivery technique where a single radiofrequency generator (e.g., power source) alternates power output between multiple different channels in a controlled time sequence or method, thus allowing each channel to receive power during designated time slots or intervals. As such, the RF generator 20 can provide RF power to the channels during assigned specific time intervals in a sequential or cyclical order. By way of example, during a designated time slot, RF power is delivered by the RF generator 20 exclusively to a selected channel, thus avoiding overlap in RF power delivery to multiple channels. In radiofrequency ablation systems, this approach enables a single generator to provide different power levels to multiple RF probes by controlling both the timing of power delivery and the voltage amplitude. The power-time multiplexing technique also enhances the efficiency, control, and management of power distribution over multiple channels when the delivery of power to both channels would otherwise exceed the total available power of the RF generator or the delivery of power to a selected channel would otherwise be constrained and unable to meet the power needs of the channel.

Time multiplexing (or power-time multiplexing) helps tissue ablation systems that utilize a single power source with limited power and time resolution, where simultaneous ablation therapies require different power levels that cannot be independently controlled through traditional control methods and techniques. The power requirements of multiple therapies performed by different RF probes coupled to a single RF generator can be challenging during various phases of therapy, such as the initial thermal ramping period, temperature maintenance, or recovery after high impedance events.

By implementing time multiplexing control, the tissue ablation system 10 of the present invention can deliver different power levels to each channel while using a common voltage amplitude. This is accomplished by alternating the RF power output between the channels and controlling the duration of power delivery to each channel within a defined cycle period or duty cycle. The power-time multiplexing technique enables power delivery that satisfies the following requirement:

PowerCh1+PowerCh2≤Total Available Power
where PowerCh1 (power supplied to channel 1) and PowerCh2 (power supplied to channel 2) are the power amounts demanded by the first and second channels respectively, which when summed do not exceed the total power available from the RF generator.

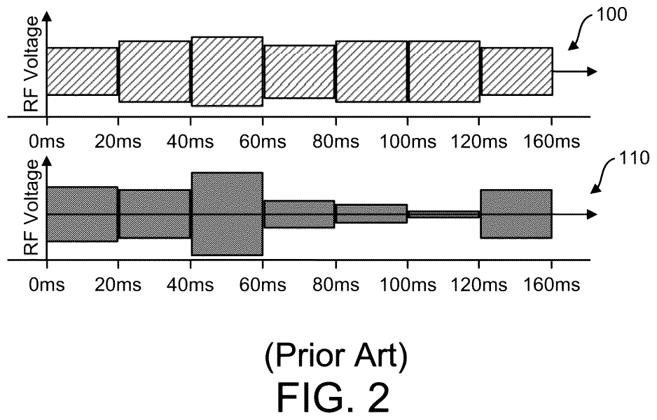

FIG. 2 illustrates by simple way of example conventional operating power profiles of two independent therapies performed over separate channels when each RF probe is coupled to a dedicated, or separate, RF generators. The upper graph 100 shows an RF voltage profile over time for a first channel formed by a first RF probe coupled to a first RF generator over a selected period of time (e.g., a 160 millisecond (ms) period). The lower graph 110 shows an RF voltage profile over time for a second channel formed by a second RF probe coupled to a second different RF generator over the same time period. Each channel operates independently of each other, with each channel having a specific voltage amplitude related to the type of therapy being performed and timing characteristics determined by the specific therapy requirements. The illustrated voltage and time profiles represent a case where each channel can receive exactly the power required to perform the specific therapy at any given time, without applied power constraints from sharing a common power source.

The illustrated voltage graphs 100, 110 demonstrate how different therapy requirements require different voltage levels and time durations over the course of the procedure. For example, the first graph 100 associated with the first channel shows modest changes in RF voltage levels during each time interval (e.g. pulse widths) over the course of the time or cycle period. The second channel exhibits more varied RF voltage levels and pulse patterns over the cycle period. This independent operation ensures that each channel receives the necessary RF power from the RF generator, without consideration for the other channel's power needs.

This independent channel operation serves as a reference point for understanding the challenges that need to be addressed when implementing a power-time multiplexing technique when utilizing a single RF generator. The tissue ablation system 10 can be configured to approximate the dual independent therapy required power profiles while utilizing a single power source.

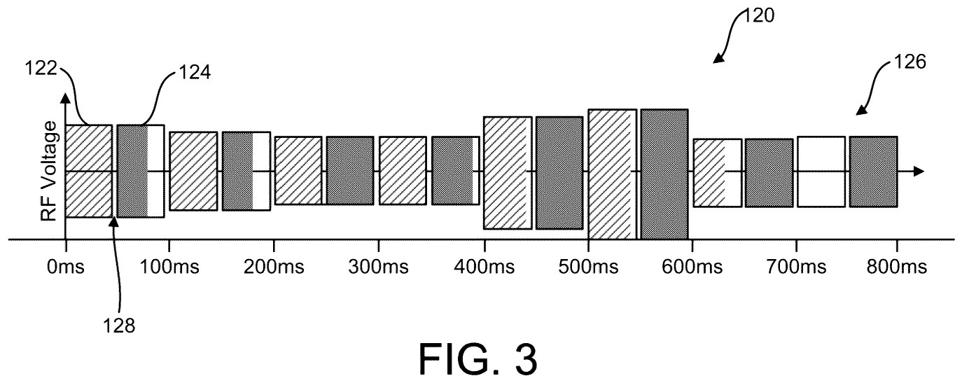

FIG. 3 is a graph 120 showing the power-time multiplex technique used to deliver RF energy to multiple (e.g., two) channels from a single RF generator 20 according to the teachings of the present invention. The controller 24 can be programmed to implement the power-time multiplexing techniques described herein. The illustrated graph 120 graphically displays RF voltage versus time over a selected cycle period (e.g., a 800 ms period). The graph 120 shows the alternating delivery of power to a first channel 122 and to a second channel 124 during discrete time intervals, which correspond to duty cycles. According to one embodiment, the duty cycle time can be fixed or predetermined. In the illustrated graph 120, the average power delivered to each channel is determined by selected factors, such as for example the time duration of RF power supplied on each channel and the RF voltage amplitude.

The relationship between average power, peak power, and time can be expressed as:

Power avg=Power peak×(time percent)

In the time multiplex technique employed by the RF generator 20 of the present invention, the duty cycle represents a fraction of time within each cycle period during which RF power can be delivered to a specific channel. For example, in a 100 ms cycle period, if a channel receives RF power for 40 ms, the duty cycle is 40%. The duty cycle can hence directly affect the average power delivered to each channel, where a higher duty cycle results in higher average power delivery, while a lower duty cycle reduces the average power delivered to the channel.

When implementing the power-time multiplexing techniques of the present invention with a single RF generator 20, the amplitude of the RF voltage supplied by the generator 209 can be delivered to whichever channel requires the higher power levels. The timing of the delivery of RF power to each channel can then be adjusted to achieve the desired average power for that channel. For example, if one channel requires 30 W average RF power and another channel requires 15 W average RF power, assuming the same voltage amplitude, then the duty cycle of the second channel is half that of the first channel.

The fixed power-time multiplexing technique illustrated by the graph 120 shown in FIG. 3 illustrates how RF voltage is delivered in alternating periods between first and second channels during fixed duty cycles. Each channel receives power for a portion or all of the overall cycle time, with the cycle time portions determined by the calculated duty cycles needed to achieve the desired power levels. In the illustrated example, the cycle period 126 can be 800 ms or longer and a cycle time of about 100 ms, and the duty cycle for each channel portion of the cycle time can be about 50 ms. For the illustrated cycle time, the combined duty cycles of both channels are less than 100% to account for switching time between the two channels. As shown in graph 120, the first channel 122 can have RF power delivered thereto for a selected portion of a defined duty cycle (e.g., 50 ms). In the illustrated embodiment, the RF power is provided for the complete duty cycle at a selected RF voltage level. With regard to the second channel 124, which can be used to perform a separate therapy, the RF power can be delivered to the second channel for a portion of the duty cycle (e.g., less than 50 ms), also at a defined RF voltage level. For the cycle time portion of the cycle period 126 between 100 ms and 200 ms, the RF voltage levels can be different relative to the first cycle time portion, and again the RF power associated with the second channel can be less than the duty cycle (e.g., 50 ms).

The voltage profile of the graph 120 repeats the foregoing power time pattern throughout the therapy delivery period. As tissue impedance changes or power requirements shift during therapy, the duty cycles for each channel can be recalculated and adjusted while maintaining the same RF voltage amplitude. This enables dynamic control of average power delivery to each channel without requiring changes to the RF voltage level.

The power-time multiplexing technique of the present invention also considers limitations in switching between the channels. For example, each transition between channels requires a selected amount of time for the RF output to stabilize. The transition periods are represented by time gaps 128 between the voltage pulses associated with the first channel 122 and the second channel; 124. The system 10 can account for the transition times when calculating duty cycles to enable accurate power delivery to each channel.

The regular switching pattern shown in FIG. 3 represents one possible implementation of the power-time multiplexing technique of the present invention. The duration and spacing of the voltage pulses can be modified to accommodate different power requirements while maintaining the relationship between peak power, duty cycle, and average power delivery.

The fixed power-time multiplexing technique can also account for scenarios where the combined pulse widths of both channels exceed the available cycle time. When the sum of both channel pulse widths is greater than the predetermined or fixed cycle period (100 ms-8 ms), where 8 ms accounts for required switching time, the controller 24 can determine new duty cycles and cycle times to allow for RF power to be delivered to the channels within the predefined duty cycles while maintaining proper power delivery ratios.

According to one embodiment, determination of the duty cycles and cycle times by the controller 24 can be expressed as:

DAC1/sqrt(duty1)=DAC2/sqrt(1−0.08−duty1);
where DAC1 and DAC2 represent the power values for each channel, duty1 is the duty cycle of the first channel, and 0.08 represents the 8 ms switching time requirement expressed as a percentage of the 100 ms cycle time. In the current example, the minimum duty cycle of a given channel is 2 ms/100 ms, where 2 ms represents the minimum pulse width.

For situations where the input pulse width of a given channel is not 100 ms, the controller 24 can determine the average DAC value for the duty cycle using the foregoing formula. For example, if the input duty cycle of the first channel is not 100%, then:

DAC1=DAC1input×sqrt(PW1/100 ms); where PW1 represents the pulse width for the first channel.

These calculations enable precise control of RF power delivery while accommodating system timing constraints. The duty cycle adjustments maintain the desired power ratios between channels while operating within the physical limitations of the RF generator and switching circuitry. When pulse widths of the delivered RF power can be adjusted, the system 10 preserves the relative power delivery requirements of each channel by maintaining proportional relationships in the duty cycle calculations.

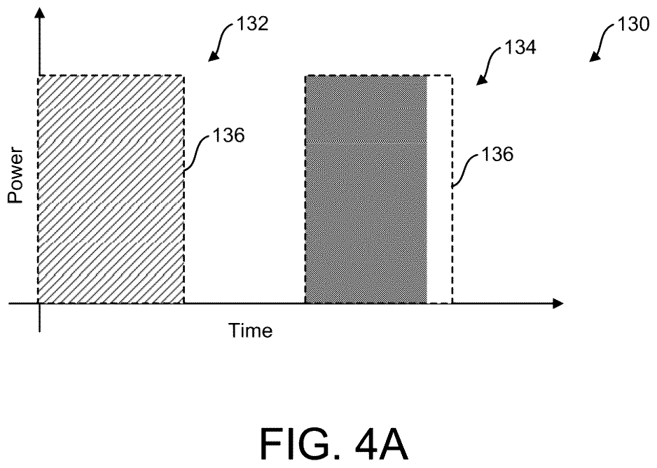

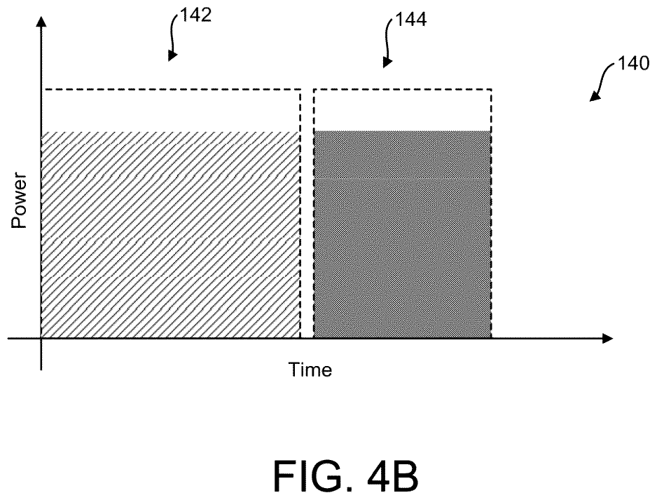

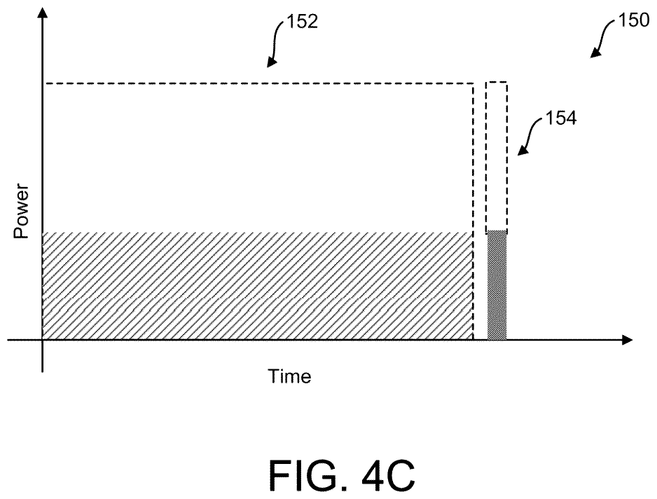

The fixed power-time multiplexing technique described thus far represents a baseline implementation where the channels alternate within fixed or predetermined duty cycles and cycle periods. However, additional optimization of power-time can be achieved through the implementation of additional power-time rebalancing techniques, which adjust the distribution of RF power delivery time between channels. These power rebalancing techniques, illustrated in FIGS. 4A-4C, provide methods and techniques to further optimize RF voltage amplitude and RF power delivery efficiency.

FIGS. 4A-4C illustrate example power-time rebalancing techniques employed as part of or in addition to the power-time multiplexing techniques implemented by the RF generator 20 of the present invention. The power-time rebalancing technique can redistribute power delivery time between channels while reducing the required RF voltage amplitude.

The initial RF power for each channel is calculated by the controller 24 based on a PID (Proportional-Integral-Derivative) output:

P1=(V1×V1/R1)×D1=Varg1×Varg1/R1;
where Varg1=V1×sqrt(D1);

P2=(V2×V2/R2)×D2=Varg2×Varg2/R2; and
where Varg2 =V2×sqrt(D2).

In the foregoing equations, V1 and V2 represent the initial RF voltages of each channel, R1 and R2 represent the channel resistances, and D1 and D2 represent the duty cycles for each channel respectively.

FIG. 4A shows a graph 130 of the RF power distribution over multiple channels before power-time rebalancing by the controller 24. The first channel and the second channel are allocated sequential time segments. The graph 130 displays power versus time distribution for the first channel 132 and the second channel 134 based on the foregoing PID calculations. The dashed lines 136 indicate the total available duty cycle time for power delivery for each channel. In the current example, the duty cycles are the same.

FIG. 4B illustrates the effects of the power-time rebalancing technique implemented by the RF generator 20 of the present invention. Through rebalancing of the time and power allocated to each channel, the generator 20 maximizes the usage of available time while maintaining sufficient average power levels to perform the therapies associated with each channel. In the illustrated example, a graph 140 of power over time shows the rebalanced or adjusted duty cycle and RF power associated with the first channel 142 and the second channel 144. The duty cycle associated with the first channel has been increased to allow for the delivery of additional RF power while maintaining a similar or constant RF voltage, and the corresponding duty cycle associated with the second channel has been decreased to provide for the delivery of less power while maintaining a similar or constant RF voltage. As such, the power-time rebalancing technique employed by the controller 24 can reallocate or rebalance duty cycle time to one of the channels to provide for additional RF power as the need arises. As such, the power-time rebalancing technique seeks to scavenge (by reallocation) time and hence power from one channel for use by the other channel. The rebalanced power calculations follow:

P1=(Vnew×Vnew/R1)×Dnew1=Varg1×Varg1/R1

P2=(Vnew×Vnew/R2)×(0.92−Dnew1)=Varg2×Varg2/R2

The new power-time rebalancing (e.g., duty cycle and RF voltage) can be determined by:

Dnew1=Varg1×Varg1/(Varg1×Varg1+Varg2×Varg2)

Vnew=Varg1/sqrt(Dnew1)

The foregoing equations ensure that the power delivered to each channel remains consistent with the power requirements of the therapies while utilizing reduced RF voltage amplitudes. The factor 0.92 represents the maximum available percentage (92%) of a predefined duty cycle or cycle time after accounting for switching time requirements.

By extending or adjusting the time allocated for RF power delivery for one channel relative to another channel (e.g., adjusting the duty cycles) according to the foregoing formulas, the RF generator 20 can maintain equivalent RF power delivery using lower voltage amplitudes. This power-time rebalancing reduces the voltage requirements while preserving the ability to effectively perform the therapies associated with each channel.

FIG. 4C illustrates a specific case where one channel requires active therapy while the other channel is disposed in an inactive state. Specifically, FIG. 4C shows a graph 150 having a first active channel 152 having a defined duty cycle, RF power amount, and RF voltage level, and a second inactive channel 154. The power-time rebalancing technique has been applied to provide for an extended duty cycle for channel 1. In this scenario, the power distribution allocates most of the cycle time to the active channel (e.g., first channel), represented by the longer power delivery duty cycle. The inactive channel receives minimal RF power sufficient only for monitoring purposes, shown by the relatively small duty cycle.

The power delivery requirements for each channel can vary throughout the procedure. For example, the inactive channel can require only monitoring pulses (e.g., 2 ms duty cycle), while the active channel can utilize the majority of the available cycle time. This enables further RF voltage amplitude reduction while maintaining the amount of RF power delivered to the active channel.

The power-time rebalancing calculations continuously adjust the cycle time and duty cycles assigned to each channel based on real-time power demands, tissue impedance measurements and changes, and therapy requirements. This dynamic adjustment performed by the power-time rebalancing technique enables optimal power delivery while maintaining the lowest RF voltage amplitude throughout the procedure and enables the generator 20 to handle disparate power differences between the channels.

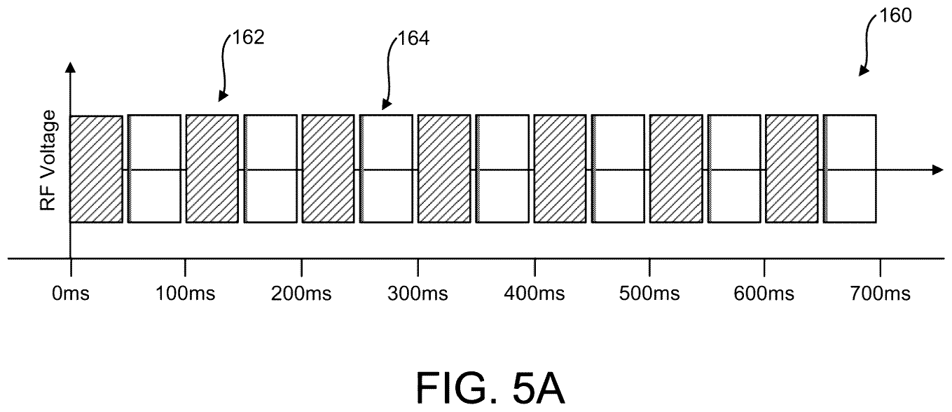

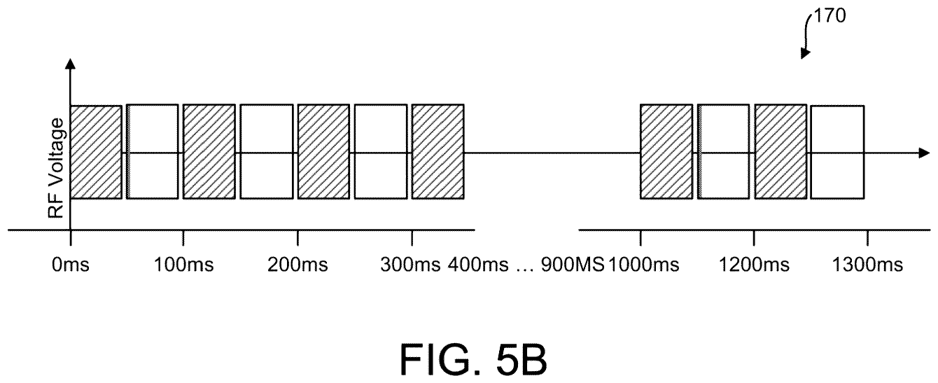

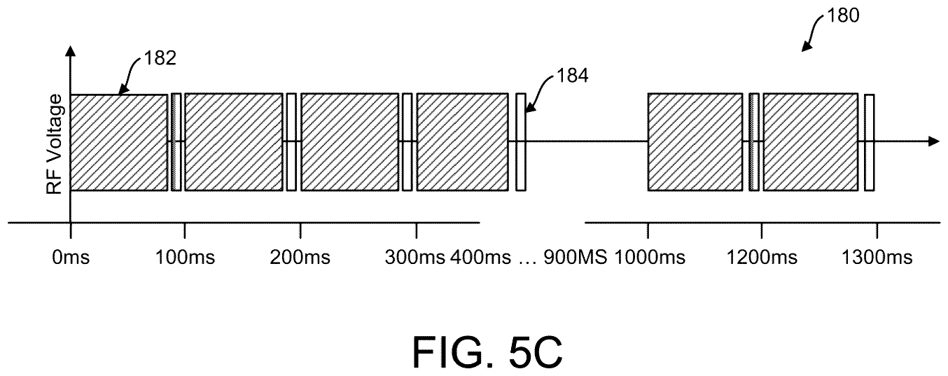

The power-time rebalancing technique also forms the basis for the specific voltage delivery patterns demonstrated in FIGS. 5A-5C, which show the implementation of these calculations in various therapeutic scenarios.

FIGS. 5A-5C illustrate various RF voltage delivery patterns implemented through the power-time multiplexing technique, demonstrating how the theoretical power calculations occur in actual power delivery timing patterns.

For example, FIG. 5A shows a graph 160 illustrating the RF voltage delivery pattern over multiple channels 162, 164 during regular multiplex timing between channels over a cycle period of 700 ms period and having a predetermined or fixed duty cycle per channel. Each time segment represents an RF voltage pulse, with alternating power delivery between the first and second channels. The voltage pulses demonstrate a baseline implementation where:

Pavg=((100×100)/200)×(2/100)=1.0 Watt;
where 100V represents the RF output, 200 Ω represents the impedance, and the ratio 2/100 represents the duty cycle for impedance measurement. The regular spacing between pulses allows for consistent RF power delivery to each channel while maintaining the calculated duty cycles for each channel.

FIG. 5B shows a graph 170 that depicts RF voltage delivery with extended gaps between active channel periods, spanning for example a cycle period of about 1300 ms. The pattern illustrated in the graph 170 emerges when measuring inactive channel impedance during selected duty cycles (e.g., every ten duty cycles), resulting in:

Pavg=((100 ×100)/200)×(2/1000)=0.1 Watt

The extended time gaps between power pulses demonstrates how the RF generator 20 maintains monitoring capability while reducing overall power delivery to the channels not requiring power for therapy. The illustrated timing pattern preserves the impedance measurement capability of the generator 20 while minimizing energy delivery during inactive periods. The illustrated cycle period employs predefined cycle times having predefined duty cycles of equivalent sizes for each channel.

FIG. 5C shows a graph 180 that illustrates RF voltage delivery with minimized switching periods between multiple channels, also spanning a cycle period of about 1300 ms. Specifically, the graph 180 has a first channel duty cycle 182 that is greater than the duty cycle 184 of a second channel. The different duty cycles for each channel can be generated and implemented using the power-time rebalancing technique described herein. In this example implementation, the RF generator 20 maintains an impedance pulse width and rate at selected defined intervals (e.g., every 2 ms) during the second duty cycle 184, while utilizing most of the duty cycle 182 for the active channel. This pattern optimizes power delivery by maximizing time allocation to the active therapy channel, maintaining minimal but sufficient power delivery for impedance monitoring, preserving the capability to detect changes in tissue conditions, and minimizing switching between the channels.

The voltage delivery patterns shown in these figures correspond to different operational scenarios while following the power-time rebalancing and multiplexing calculations described herein. The tissue ablation system 10 dynamically selects and implements these patterns based on active therapy requirements for each channel, impedance monitoring needs, power delivery efficiency considerations, and overall system timing constraints.

These patterns demonstrate how the time multiplexing strategy adapts to various therapy scenarios while maintaining control over power delivery to each channel. The timing patterns can be dynamically adjusted throughout the procedure as channel requirements change, enabling optimal power delivery while operating within system constraints.

In some embodiments, the RF generator 20 can employ a fixed 50/50 power-time multiplexing technique for power multiplexing between two channels having similar or identical predefined duty cycles. For example, the RF generator 20 can divides each cycle period (e.g., 100 ms) equally between the channels to predetermined duty cycles (e.g., 46 ms), while concomitantly allocating time to each channel (e.g., 4 ms) reserved for switching between the channels. The voltage amplitude for both channels is set according to whichever channel requires higher power delivery. For example, if the first channel requires 30 W and the second channel requires 15 W, then the RF voltage amplitude is set based on the requirements of the first channel. The second channel then achieves a lower power requirement by utilizing the same voltage amplitude as the first channel but potentially not requiring the entire duty cycle to deliver an equivalent amount of RF power.

In some embodiments, the tissue ablation system 10 of the present invention can implement dynamic power-time rebalancing of the duty cycle allocation for each channel. When the combined power demands of both channels can be satisfied with lower voltage amplitude by adjusting the time allocation, the system 10 can determine the new duty cycles according to:

Dnew1=Varg1×Varg1/(Varg1×Varg1+Varg2×Varg2);

Vnew=Varg1/sqrt(Dnew1);
where Varg1 and Varg2 represent the adjusted voltages for each channel. The new duty cycle Dnew1 determines the portion of the cycle allocated to channel one, while channel two receives the remaining time up to 92% of the cycle (accounting for switching time). For instance, if channel one requires twice the power of channel two, it might receive 60% of the cycle time while channel two receives 32%.

In an embodiment with a single active channel, the system can allocate most of the cycle time to the channel requiring active therapy while maintaining minimal power delivery to the inactive channel. The inactive channel receives brief power pulses, typically 2 ms in duration, sufficient for impedance monitoring. This enables the system to reduce voltage amplitude while maintaining full therapy delivery to the active channel and preserving monitoring capability on the inactive channel.

The system can also implement various impedance measurement timing strategies. In some embodiments, impedance measurements can occur every duty cycle using 2 ms pulses, resulting in an average power of 1.0 Watt during measurement (calculated as ((100V ×100V)/200 Ω)×(2 ms/100 ms)). In some embodiments, measurements can occur every ten duty cycles, reducing average power to 0.1 Watt during measurement periods. The system selects between these measurement strategies based on therapy requirements and the need for impedance monitoring frequency.

These embodiments demonstrate how the time multiplexing strategy adapts to various therapy scenarios while maintaining precise control over power delivery to each channel. The timing patterns can be adjusted throughout the procedure as channel requirements change, enabling optimal power delivery while operating within system constraints.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

1. A tissue ablation system, comprising:
a radiofrequency generator having a power source and a controller operatively coupled to the power source and having a processor and a memory element;
a first radiofrequency probe having at least one electrode and electrically coupled to the radiofrequency generator to form a first channel;
a second radiofrequency probe having at least one electrode and electrically coupled to the radiofrequency generator to form a second channel;
wherein the power source is configured to deliver radiofrequency power to the electrodes of the first and second radiofrequency probes sufficient to provide controlled heating of tissue surrounding the electrodes during a surgical procedure;
wherein the processor is configured to execute program instructions to:
determine a first power demand for the first channel and a second power demand for the second channel;
implement a power-time multiplexing technique by:
allocating a first duty cycle to the first channel and a second duty cycle to the second channel; and
determining a common radiofrequency voltage for each of the first channel and the second channel during the first and second duty cycles, respectively, based on whichever of the first power demand or the second power demand has a higher radiofrequency voltage; and
deliver radiofrequency power at the common radiofrequency voltage between the first channel and the second channel within each of the first and second duty cycles, respectively, when each of the first and second channels is in an activation state.

2. The tissue ablation system of claim 1, wherein the first duty cycle and the second duty cycle have the same time duration.

3. The tissue ablation system of claim 2, wherein during the power-time multiplexing technique the processor is further configured to:
determine a first average power for the first channel according to the formula P1=(V1×V1/R1)×D1, wherein V1 is voltage, R1 is impedance, and D1 is duty cycle for the first channel; and
determine a second average power for the second channel according to the formula P2=(V2×V2/R2)×D2, where V2 is voltage, R2 is impedance, and D2 is duty cycle for the second channel.

4. The tissue ablation system of claim 1, wherein the processor is further configured to implement a power-time rebalancing technique by adjusting a duration of each of the first duty cycle and the second duty cycle based on the respective power demands of the first channel and the second channel.

5. The tissue ablation system of claim 4, wherein the processor is configured to, during the power-time rebalancing technique:
determine a new first duty cycle (Dnew1) for the first channel according to Dnew1=Varg1×Varg1/(Varg1×Varg1+Varg2×Varg2), wherein Varg1 and Varg2 are adjusted voltages for the first and second channels, respectively; and
determine a new radiofrequency voltage amplitude (Vnew) during the new first duty cycle according to Vnew=Varg1/sqrt(Dnew1).

6. The tissue ablation system of claim 4, wherein the processor is further configured to allocate up to 92% of a cycle period between the first and second channels.

7. The tissue ablation system of claim 1, wherein the processor is further configured to:
monitor an impedance of each of the first and second channels during the respective activation state; and
adjust an amplitude of the common radiofrequency voltage based on the impedance.

8. The tissue ablation system of claim 1, wherein the processor is configured to adjust a duration of one of the first duty cycle and the second duty cycle during the activation state to maintain equivalent radiofrequency power delivery thereto while reducing an amplitude of the common radiofrequency voltage.

9. The tissue ablation system of claim 1, wherein the first duty cycle and the second duty cycle define a cycle time, and wherein, if one of the first and second channels requires minimal or no power, the processor is configured to:
allocate a majority of the cycle time to the other one of the first and second channels; and
reduce an amplitude of the common radiofrequency voltage.

10. The tissue ablation system of claim 1, wherein the processor is further configured to determine a minimum common radiofrequency voltage capable of satisfying the first power demand and the second power demand of the first and second channels, respectively.

11. The tissue ablation system of claim 1, wherein the controller is further configured to:
maintain the determinations of the first and second power demands of the first and second channels, respectively; and
continuously adjust the first and second duty cycles based on changes in the first and second power demands.

12. The tissue ablation system of claim 1, wherein the controller is configured to determine the first and second duty cycles by:
determining which of the first and second channels requires a higher power demand;
allocating a larger portion of the cycle time to the duty cycle of the channel requiring the higher power demand; and
allocating a remaining portion of the cycle time to the other one of the first and second channels.

13. The tissue ablation system of claim 1, wherein the controller is configured to determine a minimum common radiofrequency voltage amplitude by:
determining the radiofrequency voltage requirements for each of the first and second channels based on associated power demands and allocated with the first and second duty cycles;
selecting a higher one of the radiofrequency voltage requirements as the common radiofrequency voltage amplitude; and
adjusting the first and second duty cycles to maintain power delivery at the selected voltage amplitude.

14. The tissue ablation system of claim 1, wherein the controller is configured to monitor power delivery to each channel during the respective activation state; detect when one of the first and second channels requires minimal power delivery; and reallocate the first and second duty cycles to minimize an amplitude of the common radiofrequency voltage.

15. The tissue ablation system of claim 1, wherein the controller is configured to determine average powers for the first and second channels based on the common radiofrequency voltage and their respective duty cycles.

16. The tissue ablation system of claim 1, wherein the controller is configured to maintain equivalent power delivery while reducing the common radiofrequency voltage by increasing the duty cycle allocated to the channel requiring higher power.

17. A computer-implemented method of allocating power in a tissue ablation system having a radiofrequency generator and first and second radiofrequency probes forming respective first and second channels, the method comprising:
executing, by a processor of a controller operatively coupled to the radiofrequency generator, program instructions stored in a memory element to:
determine a first power demand for the first channel and a second power demand for the second channel;
implement a power-time multiplexing technique by:
allocating a first duty cycle to the first channel and a second duty cycle to the second channel; and
determining a common radiofrequency voltage for each of the first channel and the second channel during the first and second duty cycles, respectively, based on whichever of the first power demand or the second power demand has a higher radiofrequency voltage; and
deliver radiofrequency power at the common radiofrequency voltage between the first channel and the second channel within each of the first and second duty cycles, respectively, when each of the first and second channels is in an activation state.

18. The computer-implemented method of claim 17, wherein during the power-time multiplexing technique the processor is further configured to:
determine a first average power for the first channel according to the formula P1=(V1×V1/R1)×D1, wherein V1 is voltage, R1 is impedance, and D1 is duty cycle for the first channel; and
determine a second average power for the second channel according to the formula P2=(V2×V2/R2)×D2, where V2 is voltage, R2 is impedance, and D2 is duty cycle for the second channel.

19. The computer-implemented method of claim 17, wherein the processor is further configured to implement a power-time rebalancing technique by adjusting a duration of each of the first duty cycle and the second duty cycle based on the respective power demands of the first channel and the second channel.

20. The computer-implemented method of claim 19, wherein the processor is configured to, during the power-time rebalancing technique:
determine a new first duty cycle (Dnew1) for the first channel according to Dnew1=Varg1×Varg1/(Varg1×Varg1+Varg2×Varg2), wherein Varg1 and Varg2 are adjusted average voltages for the first and second channels, respectively; and
determine a new radiofrequency voltage amplitude (Vnew) during the new first duty cycle according to Vnew=Varg1/sqrt(Dnew1).
